(19) United States
(12) Patent Application Publication
(10) Pub. No.: US 2023/0404865 A1
(43) Pub. Date: Dec. 21, 2023

(54) TOPICAL SKIN CARE COMPOSITION

(71) Applicant: The Procter & Gamble Company, Cincinnati, OH (US)

(72) Inventors: Thomas Elliot Rabe, Baltimore, MD (US); Benjamin Parker Heath, Loveland, OH (US); Shawn David McConaughy, Cincinnati, OH (US); Sydnee Redana Ruff, Cincinnati, OH (US)

(21) Appl. No.: 18/335,370

(22) Filed: Jun. 15, 2023

Related U.S. Application Data
(60) Provisional application No. 63/352,271, filed on Jun. 15, 2022.

(51) Int. Cl. A61K 8/06; A61K 8/73; A61K 8/49; A61K 8/9789; A61K 8/92; A61Q 19/00

(52) U.S. Cl. CPC A61K 8/062; A61K 8/732; A61K 8/4926; A61K 8/9789; A61K 8/922; A61Q 19/007

(57) ABSTRACT

A skin care composition is an emulsion having a continuous phase and a dispersed phase. The continuous phase includes water and glycerin. The dispersed phase includes at least two emollients. The composition also includes a feel modifying particle and an emulsifier. IN some examples, the feel modifying particle can include modified corn starch and colloidal oatmeal. The emulsifier is cetearyl olivate and sorbitan olivate. The composition can be used to soothe irritated skin; provide relief to itchy, dry, irritated skin; and/or moisturize and soften skin.

### FIELD OF THE INVENTION

The present disclosure relates to a topical skin care emulsion composition that can contain relatively high levels of glycerin and two or more emollients. The present disclosure further relates to methods of soothing irritated skin; relieving itchy, dry, irritated skin; and moisturizing to soften skin using such topical skin care compositions.

### BACKGROUND OF THE INVENTION

Skin can become itchy, red, flakey, dry, cracked, scaley, tight, irritated and/or rough. These skin conditions can be caused by eczema, psoriasis, and/or xerosis. These conditions can be multifactorial in etiology and may occur as a response to changes in the environment (e.g., cold weather or an immune trigger such as an allergen response), underlying diseases, medications, and/or age. Such skin conditions may be acute in nature (i.e., occur in a singular, independent incidence) or may be more chronic (i.e., affect the individual for a prolonged period of time). When symptoms of unhealthy skin occur, the person suffering from the condition generally wishes to alleviate the symptoms as quickly as possible.

Topical moisturizers can be used to treat challenged, dry, and/or irritated skin and the most effective moisturizers can have a relatively high level of glycerin and/or emollient(s). However, the use of high levels of glycerin and/or emollients can result in trade-offs with pleasant skin feel with many effective moisturizers feeling heavy and/or tacky, which is generally not consumer preferred. Instead, consumers want moisturizers that are highly efficacious and have a silky texture that seems to absorb quickly into the skin. It can also be difficult to emulsify compositions with high levels of glycerin and/or emollient(s), leading to phase instability, which can appear as but is not limited to an oily layer that has separated from the moisturizer.

Therefore, there is a need for a topical skin care product that contains a relatively high level of glycerin and/or emollient(s) that is phase stable and has low tack.

### SUMMARY OF THE INVENTION

A skin care composition comprising: (a) a continuous phase comprising: (i) from about 45 to about 85 wt. % water; (ii) from about 4 to about 18 wt. % glycerin; (b) a dispersed phase comprising at least two waxy emollients comprising: (i) from about 0.5 to about 10 wt. % of an ester comprising from about 10 to about 28 carbon atoms; (ii) from about 0.1 to about 5 wt. % of a fatty alcohol wherein the fatty alcohol comprises from about 12 to about 22; and (c) from about 0.1 to about 5 wt. % of a feel modifying particle chosen from siloxane, starches, colloidal oatmeal, or mixtures thereof; and (d) 2 wt. % or less of an emulsifier comprising cetearyl olivate and sorbitan olivate.

A skin care composition comprising: (a) a continuous phase comprising: (i) from about 55 to about 85 wt. % water; (ii) from about 5 to about 12 wt. % glycerin; (b) a dispersed phase comprising: (i) from about 1 to about 15 wt. % of at least two waxy emollients chosen from cetyl alcohol, glyceryl stearate, petrolatum, shea butter, or mixtures thereof; (ii) from about 3 to about 10 wt. % of a liquid emollient chosen from dimethicone, isopropyl isostearate, or mixtures thereof; (a) from about 0.1 to about 3 wt. % of colloidal oatmeal; (b) from about 0.1 to about 2 wt. % of an emulsifier comprising cetearyl olivate and sorbitan olivate.

### BRIEF DESCRIPTION OF THE DRAWINGS

While the specification concludes with claims particularly pointing out and distinctly claiming the subject matter of the present invention, it is believed that the invention can be more readily understood from the following description taken in connection with the accompanying drawings, in which:

FIG. 1 is a graph showing the viscosity of skin care compositions with an emollient containing either isohexadecene or triheptanoin and differing emulsifiers.

FIG. 2 is a graph of tack force vs time for Examples G-2 and H-2.

FIG. 3 is a graph showing the viscosity of skin care composition H-1 and H-2.

FIG. 4 illustrates a setup for the Tack Method.

### DETAILED DESCRIPTION OF THE INVENTION

It was found that a topical skin care composition containing a relatively high level of glycerin (e.g., from about 6.5% to about 18%) and at least two emollients in combination with an emulsifier comprising cetearyl olivate and sorbitan olivate can be phase stable and can be effective at treating challenged, dry, and/or irritated skin, while also having a consumer preferred silky texture that seems to absorb quickly into the skin and does not feel too heavy. The aesthetic properties and in some instances the efficacy can be further improved by adding siloxane, starches, and/or colloidal oatmeal to the skin care composition.

Emollients can soften, moisturize, and/or protect the skin against irritation as well as aid in solubility of various skin care benefit ingredients. However, emollients can have a range of polarity, which can make it difficult to select an effective emulsifier.

Also, it can be advantageous for the emulsifier to be efficient, since emulsifiers are generally used at the lowest level that gives the desired product viscosity and stability to give the best product performance. If an emulsifier is only effective when used at a relatively high level, the product may be difficult to spread and instead of absorbing into the skin, the product can remain as a white layer on top of the skin. In these cases, the product can also feel heavy and/or tacky. In addition, it is preferable to utilize emulsifiers which further do not also add to undesirable skin feel by imparting waxy, heavy and/or coated skin feel after application.

Tack force generally refers to the peak force required to separate an object from the composition after contacting the composition. At least some users associate good moisturization with a high tack force, especially during and/or shortly after application of the product (e.g., less than 10, 9, 8, 7, 6, 5, 4, 3, 2, or even less than 1 minute after application). However, an increase in tack force is typically accompanied by an increase in stringiness and the moisturizer feels heavy on top of the user's skin. And while a user may tolerate or even desire some stringiness and/or heaviness during and/or immediately after application as a signal of moisturization, it may be undesirable for the stringiness to persist, for example, more than 1, 5, 7, 10, or even more than 20 minutes after application of the product.

The composition can have an average tack force between 20 and 40 minutes of less than 750 g, alternatively less than 725 g, alternatively less than 710 g, and alternatively less than 705 g. The composition can have an average tack force between 20 and 40 minutes from about 250 g to about 750 g, alternatively from about 400 g to about 725 g, alternatively from about 500 g to about 710 g, and alternatively from about 550 g to about 705 g.

The composition can be opaque. The composition can have a percent transparency (% T) of less than 70%, alternatively less than 60%, alternatively less than 50%, alternatively less than 40%, alternatively less than 30%, alternatively less than 20%, alternatively less than 10%, alternatively less than 5% transmittance at 600 nm according to the % Transmittance Method, described hereafter.

Furthermore, the composition can be a physically stable emulsion. A composition is physically stable if by visual detection there is no phase separation or the viscosity varies by less than 40% when stored in a glass jar closed finger tight between t=0 (i.e., immediately after the composition is made) and a freeze thaw cycle where the sample is held at −18° C. for 24 hours then returned to room temperature or one month storage at 40° C. followed by return to room temperature. As used herein, “visual detection” means that a human viewer can visually discern if the product had separated into 2 distinct layers in a 2 oz clear glass jar with the unaided eye (excepting standard corrective lenses adapted to compensate for near-sightedness, farsightedness, or stigmatism, or other corrected vision) in lighting at least equal to the illumination of a standard 100-watt incandescent white light bulb at 30 cm.

### Definitions

As used herein, “improve the appearance of” means providing a measurable, desirable change or benefit in skin appearance, which may be quantified, for example, by a decrease in redness, inflammation, and/or plaque scales.

As used herein, “molecular weight” refers to weight average molecular weight unless otherwise stated. Molecular weights are measured using the industry standard method of gel permeation chromatography (“GPC”).

As used herein, “skin care” means regulating and/or improving a skin condition. Some nonlimiting examples include improving skin appearance and/or feel by providing a smoother, more even appearance and/or feel; increasing the thickness of one or more layers of the skin; improving the elasticity or resiliency of the skin; improving the firmness of the skin; and reducing the oily, shiny, and/or dull appearance of skin, improving the hydration status or moisturization of the skin, improving the appearance of fine lines and/or wrinkles, improving skin exfoliation or desquamation, plumping the skin, improving skin barrier properties, improve skin tone, reducing the appearance of redness or skin blotches, soothing irritated skin feel and/or improving the brightness, radiancy, or translucency of skin; preventing damage to skin via antioxidant approaches, including UV A and UV B induced damage, preventing formation of comedones, balancing the skin microbiome or preventing acne.

As used herein, “skin care active” means a compound or combination of compounds that, when applied to skin, provide an acute and/or chronic benefit to skin or a type of cell commonly found therein. Skin care actives may regulate and/or improve skin or its associated cells (e.g., improve skin elasticity, hydration, skin barrier function, and/or cell metabolism).

As used herein, “skin care composition” means a composition that includes a skin care active and regulates and/or improves skin condition.

As used herein, “substantially free” means less than 0.5%, less than 0.25%, less than 0.1%, less than 0.05%, and/or less than 0.01%.

As used herein, “treatment period,” means the length of time and/or frequency that a material or composition is applied to a target skin surface.

As used herein, “viscosity” means the viscosity at about 0.10 s⁻¹ as determined by the Rheology Test Method, described hereafter.

As used herein, “Brookfield viscosity” means the viscosity as determined by the Brookfield Viscosity Test Method, described hereafter.

All percentages are by weight of the cosmetic composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 21° C. and at ambient conditions, where “ambient conditions” means conditions under about 1 atmosphere of pressure and at about 50% relative humidity. All weights as they pertain to listed ingredients are based on the active level and do not include carriers or by-products that may be included in commercially available materials, unless otherwise specified. All numeric ranges are inclusive of narrower ranges; delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

### Composition

The skin care composition can be a stable emulsion with a continuous aqueous phase (e.g., an oil-in-water or water-in-oil-in-water emulsion) or a continuous oil phase (e.g., water-in-oil or oil-in-water-in-oil emulsion) and a dispersed phase. An emulsifier can promote the formation of the emulsion and can also help maintain product stability.

In some examples, the skin care composition can be a moisturizer with a viscosity that is sufficient for it to be dispensed from a pump or through a container that is squeezed (e.g., a tube) without sloshing or easily spilling and is also thin enough so it can be easily spread across a user's skin including but not limited to the face, eye area, hands, neck, and/or other portions of the body. The viscosity at about 0.10 s⁻¹ can be greater than or equal to 550 Pa*s, alternatively greater than or equal to 600 Pa*s, alternatively greater than or equal to 1500 Pa*s, alternatively greater than or equal to 2000 Pa*s, alternatively greater than or equal to 2500 Pa*s, alternatively greater than or equal to 3000 Pa*s. The average viscosity can be from about 550 Pa*s to about 6000 Pa*s, alternatively from about 600 Pa*s to about 5500 Pa*s, alternatively from about 800 Pa*s to about 5000 Pa*s, and alternatively from about 1000 Pa*s to about 3000 Pa*s. The viscosity at about 0.10 s¹ can be determined by the Rheology Test Method, described hereafter.

The Brookfield viscosity can be from about 10 Pa*s to about 200 Pa*s, alternatively from about 12 Pa*s to about 150 Pa*s, alternatively from about 15 Pa*s to about 125 Pa*s, alternatively from about 20 Pa*s to about 100 Pa*s, alternatively from about 22 Pa*s to about 75 Pa*s. The Brookfield viscosity can be determined by the Brookfield Viscosity Test Method, described hereafter.

### Emulsifier

The skin care composition can contain an emulsifier. The preferred emulsifier can promote the formation of the emulsion, stabilize the composition, and can work with the polymer to thicken the skin care composition.

The skin care composition can contain from about 0.10% to about 2%, alternatively from about 0.2% to about 1.5%, alternatively from about 0.4% to about 1.2%, and alternatively from about 0.6% to about 0.8% of an emulsifier. The skin care composition can contain less than 2%, alternatively less than 1.75%, alternatively less than 1.5%, alternatively less than 1.25%, alternatively less than 1%, and alternatively less than 0.8% of an emulsifier.

The emulsifier can be cetearyl olivate and sorbitan olivate (commercially available as Olivem® 1000 from HallStar®).

In another example, an additional emulsifier can be used. Examples of an additional emulsifiers include but are not limited to one or more of the following:
A mixture of waxy esters made from fatty alcohols and fatty acids derived from natural sources with an average carbon chain length ranging from about C12 to about C22
A mixture of esters made from anhydrides of natural sugars or polysugars and fatty acids derived from natural sources with an average carbon chain lengths ranging from about C12 to about C22. The mixture of esters can be made from anhydrides of of natural or naturally derived sugars including polysugars and sugar alcohol. In particular, the mixture of esters can be made from anhydrides of the following natural or naturally derived sugars: glucose, sucrose, dextrose, lactose, fructose, starch, cellulose; sugar alcohols such as glycol, glycerol, erythritol, thereitol, arabitol, xylitol, ribitol, mannitol, sorbitol, dulcitol, iditol, isomalt, maltitol, lactitol, polyglycitol, or a combination thereof.

### Dermatologically Acceptable Carrier

The continuous phase can include a dermatologically acceptable carrier (which may be referred to as a “carrier”). The phrase “dermatologically acceptable carrier” means that the carrier is suitable for topical application to the keratinous tissue, has good aesthetic properties, is compatible with the actives in the composition, and will not cause any unreasonable safety or toxicity concerns. The carrier can be present at a level of from about 50% to about 95%, about 52% to about 90%, about 55% to about 85%, or, alternatively, from about 60% to about 80%, by weight of the composition.

The carrier can be in a wide variety of forms. In some instances, the solubility or dispersibility of the components (e.g., extracts, sunscreen active, additional components) may dictate the form and character of the carrier. Non-limiting examples of the product form can include dispersions and emulsions. The oil phase of the emulsion may include silicone oils, esters, ethers, triglcyerides and mixtures thereof. The aqueous phase may include water and water-soluble ingredients (e.g., water-soluble moisturizing agents, conditioning agents, anti-microbials, humectants and/or other skin care actives). In some instances, the aqueous phase may include components other than water, including but not limited to water-soluble moisturizing agents, conditioning agents, anti-microbials, humectants and/or other water-soluble skin care actives. In some instances, the non-water component of the composition comprises a humectant such as glycerin and/or other polyol(s).

In some instances, the compositions herein are in the form of an oil-in-water (“O/W”) emulsion that provides a sensorial feel that is light and non-greasy. Suitable O/W emulsions herein may include a continuous aqueous phase of more than 50% by weight of the composition, and the remainder being the dispersed oil phase. The aqueous phase may include 1% to 99% water, based on the weight of the aqueous phase, along with any water soluble and/or water miscible ingredients. In these instances, the dispersed oil phase will typically be present at less than 30% by weight of composition (e.g., 1% to 20%, 2% to 15%, 3% to 12%, 4% to 10%, or even 5% to 8%) to help avoid some of the undesirable feel effects of oily compositions. The oil phase may include one or more non-volatile oils (e.g., botanical oils and/or silicone oils). Some nonlimiting examples of oils that may be suitable for use in the present compositions are disclosed in U.S. Pat. No. 9,446,265 and U.S. Publication No. 2015/0196464.

The carrier may contain one or more dermatologically acceptable diluents. As used herein, “diluent” refers to materials in which the skin care actives herein can be dispersed, dissolved, or otherwise incorporated. Some non-limiting examples of hydrophilic diluents include water, organic hydrophilic diluents such as lower monovalent alcohols (e.g., C₁-C₄) and low molecular weight glycols and polyols, glycerol, butylene glycol, 1,2,4-butanetriol, sorbitol esters, 1,2,6-hexanetriol, ethanol, isopropanol, sorbitol esters, butanediol, ether propanol, and combinations thereof.

### Emollient

The dispersed phase can include one or more emollients. The emollient can be a waxy emollient and/or a liquid emollient. The liquid emollient can be an oil, which can include an ester, alkane, triglyceride, non-volatile silicones, and combinations thereof. In another example, the emollient can be a waxy emollient, such as a fatty alcohol, having a melting point above 40° C., alternatively above 50° C.

The skin care composition can include from about 1% to about 30%, alternatively from about 2% to about 25%, alternatively from about 3% to about 23% emollient, alternatively from about 5% to about 22%, and alternatively from about 7% to about 20% of one or more emollients. In another example, the skin care composition can contain from about 7% to about 30%, alternatively from about 9% to about 27%, alternatively from about 12% to about 25%, alternatively from about 15% to about 24%, and alternatively from about 17% to about 22% emollient. In another example, the skin care composition can contain from about 3% to about 20%, alternatively from about 4% to about 15%, alternatively from about 5% to about 10%, and alternatively from about 6% to about 9% emollient.

In some examples, the skin care composition can include a liquid emollient and a waxy emollient. The weight ratio of waxy emollient to liquid emollient can be from about 1:5 to about 3:1, alternatively from about 1:4 to about 5:2, alternatively from about 1:3 to about 2:1, and alternatively from about 1:2 to about 7:4. In another example, the weight ratio of waxy emollient to liquid emollient can be greater than about 1:1, alternatively greater than about 3:2, and greater than about 5:3. In another example, the weight ratio of waxy emollient to liquid emollient can be from about 1:1 to about 4:1, alternatively from about 3:2 to about 3:1, alternatively from about 5:3 to about 2:1. In another example, the weight ratio of waxy emollient to liquid emollient can be from about 1:5 to about 1:1, alternatively from about 1:4 to about 4:5, and alternatively from about 1:3 to about 3:5.

The composition may contain at least two emollients. A first emollient may be a waxy emollient, such as a fatty alcohol, and the second emollient may be a liquid emollient, such as a fatty ester. Fatty alcohols and fatty esters generally have different polarities, which can make it difficult to find an emulsifier capable of emulsifying both emollients. Further, it may be challenging to keep the emulsion stable over the broad range of temperatures that a consumer skin care product may encounter during its life, including shipping, handling, storage in spaces that are not climate controlled (e.g., warehouses or in a gym bag in a user's car), and/or use. In one example, the use of an emulsifier comprising cetearyl olivate and sorbitan olivate to emulsify a fatty alcohol and a fatty ester may lead to a stable emulsion with desirable feel properties including consumer preferred tack. In an example, the emulsifier comprises cetearyl olivate and sorbitan olivate, the waxy emollient is a fatty alcohol, such as ethyl alcohol, and the liquid emollient is a fatty ester, such as isopropyl isostearate.

### Liquid Emollient

The skin care composition may include from about 0.5% to about 15%, alternatively from about 1% to about 13%, alternatively from about 3% to about 11%, alternatively from about 4% to about 9%, alternatively from about 5% to about 7% liquid emollient.

The liquid emollient may be petroleum-derived such as petrolatum and/or mineral oil, or combinations thereof. The liquid emollient can be a naturally derived oil that is a plant oil. Examples of the plant oil can include, but are not limited to, palm kernel, coconut, avocado, canola, corn, cottonseed, olive, palm, hi-oleic sunflower, mid-oleic sunflower, sunflower, palm stearin, palm kernel olein, safflower, babassu oils, and combinations thereof. In one embodiment, palm kernel oil may be the selected oil. In another embodiment, coconut oil may be the selected oil. In another embodiment, the plant oil may be a combination of palm kernel oil and coconut oil.

The skin care composition can include one or more of the following liquid emollients:
An alkane or isoalkane, ranging in total carbon from about C8-C60, including but not limited to squalane, squalene, coconut alkanes, coconut/palm kernel alkanes, C9-12 Alkane, C10-13 Alkane, C13-15 Alkane, C15-19 Alkane, C14-22 Alkane, C30-34 Alkane, C9-20 Isoalkane, C10-12 Isoalkane, C12-15 Isoalkane, isohexadecene, C32-54 Isoalkane, or a combination thereof.
An ester formed from an alcohol having from 1-6 carbons and acid having from about 6-22 carbons, including but not limited to isopropyl isostearate, isopropyl palmitate, isoamyl laurate, shea butter ethyl esters, shea butter cetyl esters, shea butter decyl esters, shea butter oleyl esters, jojoba esters, coco-capylate/caprate, ethyl caprate, ethylhexyl caprate, isoamyl caprylate/caprate, argan oil isostearyl esters, babassu seed oil ethyl esters, camelina sative seed oil ethyl esters, castor/olive oil esters, coconut oil decyl esters, coconut oil ethyl esters, linseed oil ethyl esters, olive oil decyl esters, olive oil ethylhexyl esters, palm kernel oil triacetin esters, palmitoyl serine/silk amino acid methyl esters, rapeseed oil decyl esters, safflower seed oil decyl esters, sunflower seed oil cetyl esters, sunflower seed oil ethyl esters, trehalose isostearate esters, or a combination thereof.
A triglyceride having 3 carbon chains where the carbon chain length ranges from about C7-C18, including but not limited to triheptanoin, caprylic/capric triglycride, C10-19 triglycerides, caprylic/capric/lauric triglycerides, hydrogenated C12-C18 triglycerides, triethylhexanoin, or a combination thereof.
A non-volatile silicone fluid including but not limited to dimethicone, amodimethicone, or a combination thereof.

### Waxy Emollient

The skin care composition may include from about 0.1% to about 22%, alternatively from about 0.2% to about 19%, alternatively from about 0.5% to about 17%, alternatively from about 1% to about 15%, alternatively from about 1.5% to about 13%, and alternatively from about 2% to about 12% waxy emollient. In one example, the skin care composition can include from about 5% to about 20%, alternatively from about 7% to about 15%, alternatively from about 9% to about 14%, and alternatively from about 11% to about 13% waxy emollient. In another example, the skin care composition can include from about 1% to about 5%, alternatively from about 2% to about 4%, and alternatively from about 2% to about 3% waxy emollient.

One or more waxy emollients can be solids at room temperature and have an average carbon chain length ranging from about 12 to about 28, alternatively about 12 to about 22, alternatively about 12 to about 18, alternatively from about 16 to about 22, and alternatively 16 to about 18.

The skin care composition can include a waxy emollient, such as a wax, butter, fatty alcohol, or a combination thereof. Waxy emollients can include, but are not limited to, beeswax, jojoba wax, shea butter, palm kernel wax, lanolin wax, shellac wax, rice bran wax, carnauba wax, ozokerite, Cocos nucifera (coconut) butter, cocoa butter, goat butter, acrocomia aculeata seed butter, chiuri butter, carnauba, candelilla, or a combination thereof.

Fatty alcohols can include monohydric alcohols having 8-22 carbon atoms, although longer chain alcohols in excess of 30 carbons may be used. The fatty alcohols may be saturated or unsaturated. The fatty alcohols may be straight or branched. In one example, the waxy emollient may comprise straight chain, saturated fatty alcohol with a terminal hydroxyl. The fatty alcohols can include decyl alcohol, lauryl alcohol, myristyl alcohol, cetyl alcohol, stearyl alcohol, isostearyl alcohol, icosyl alcohol, behenyl alcohol, batyl alcohol, arachidyl alcohol, brassica alcohol, coconut alcohol, hydrogenated jojoba alcohol, jojoba alcohol, or a combination thereof. In some examples, the fatty alcohol can have an average carbon chain length from about 12 to about 28, alternatively from about 12 to about 22, alternatively from about 16 to about 22, alternatively from about 16 to about 18. Alternatively, the fatty alcohol can have an average chain length of from about 12 to about 18.

The waxy emollient may be an ester formed from an alcohol having from 1-6 carbons and acid having from about 10 to about 28 carbons, alternatively from about 10-22 carbons. The waxy emollient may be an ester of glycerin and one or more C10 to C22 fatty acid chains. In some examples, a waxy emollient can be glyceryl monostearate. The waxy emollient may be a monoester of glycerin and one C10 to C22 fatty acid chain. In an example, a waxy emollient is glyceryl stearate.

### Humectant

The continuous phase and/or the dispersed phase can include one or more humectants. The composition can contain from about 5% to about 20%, alternatively from about 6% to about 18%, alternatively from about 7% to about 16%, alternatively from about 7.5% to about 15%, and alternatively from about 9.5% to about 12.5% humectant. Humectants are substances which provide the skin with water-retention benefits. Humectants have an affinity to hydrogen bonds of water molecules and to skin hydrophilic molecular functionalities, and accordingly bind themselves to internal water molecules as well as skin molecules, holding water to the outside surface and upper layers of the stratum corneum, thereby increasing the overall content in the skin itself. Topical application of cosmetic products containing humectants, (for example, glycerin) can be associated with improvements in barrier function, increases in epidermal thickness, and improvements in general skin appearance.

The topical skin care composition may comprise a humectant selected from the group consisting of sugar and sugar alcohols, polyhydric alcohols, amino acids and derivatives thereof such as proline and arginine aspartate, 1,3-butylene glycol, propylene glycol and water and codium tomentosum extract, collagen amino acids or peptides, creatinine, diglycerol, biosaccharide gum-1, glucamine salts, glucuronic acid salts, glutamic acid salts, polyethylene glycol ethers of glycerin (e.g., glycereth 20), glycerin, glycerol monopropoxylate, glycogen, hexylene glycol, honey, and extracts or derivatives thereof, hydrogenated starch hydrolysates, hydrolyzed mucopolysaccharides, inositol, keratin amino acids, glycosaminoglycans, methoxy PEG-10, methyl gluceth-10, methyl gluceth-20, methyl glucose, 3-methyl-1,3-butanediol, N-acetyl glucosamine salts, polyethylene glycol and derivatives thereof (such as PEG-15 butanediol, PEG-4, PEG-5 pentaerythitol, PEG-6, PEG-8, PEG-9), pentaerythitol, 1,2 pentanediol, PPG-1 glyceryl ether, PPG-9,2-pyrrolidone-5-carboxylic acid and its salts such as glyceryl pca, saccharide isomerate, sericin, silk amino acids, sodium acetylhyaluronate, sodium hyaluronate, sodium poly-aspartate, sodium polyglutamate, sorbeth 20, sorbeth 6, sugar and sugar alcohols and derivatives thereof such as glucose, mannose and polyglycerol sorbitol, trehalose, dihydrate trehalose, triglycerol, trimethyolpropane, tris (hydroxymethyl) amino methane salts, and yeast extract, and mixtures thereof.

Nonlimiting examples of sugars and sugar alcohols can include glucose, fructose, galactose, sucrose, maltose, lactose, trehalose, erythritol, threitol, arabitol, ribitol, xylitol, mannitol, sorbitol, galactitol, iditol, volemitol, fucitol, inositol, maltitol, meso-erythritol, and/or lactitol. In an example, the sugar comprises trehalose.

Nonlimiting examples of polyhydric alcohols may include glycerin, diglycerin, glycerol, erythritol, arabitol, xylitol, ribitol, mannitol, sorbitol, galactitol, fucitol, maltitol, mannose, inositol, triethyleneglycol, sodium pyrrolidone carboxylic acid (PCA), zinc PCA and derivatives and mixtures thereof.

In an example, the humectant is glycerin. The composition can contain from about 5% to about 25%, alternatively from about 6% to about 20%, alternatively from about 7% to about 17%, and alternatively from about 7.5% to about 15% glycerin.

### Polymer

The continuous and/or the dispersed phase can include one or more polymers. The polymer can help thicken the skin care composition, as well as help provide a smooth, silky feeling during and after application to the skin.

The composition may comprise greater than 0.01%, alternatively greater than 0.05%, alternatively greater than 0.1%, alternatively greater than 0.15%, alternatively greater than 0.2%, alternatively greater than 0.3%, or alternatively greater than or equal to 0.4% polymer. The composition may comprise less than or equal to 1.6%, alternatively less than or equal to 1.4%, alternatively less than or equal to 1.3%, alternatively less than or equal to 1.0% polymer. In some examples, the composition is a moisturizer, such as a cream or lotion, and can include from about 0.01% to about 3%, alternatively from about 0.05% to about 2.5%, alternatively from about 0.1% to about 2%, alternatively from about 0.15% to about 1.5%, alternatively from about 0.2% to about 1.2%, or alternatively from about 0.25% to about 0.75% polymer.

In other examples, the composition can include sodium polyacryloyldimethyl taurate, hydroxyethyl acrylate/sodium acrloyldimethyl taurate copolymer, Sodium acrylate/Sodium Acryloyldimethyl Taurate copolymer, Ammonium Polyacryloyldimethyl taurate, Ammonium Acryloyldimethyltaurate/VP Copolymer, sodium acrylates copolymer, Polyacrylate Crosspolymer-11, Polyacrylate Crosspolymer-6, carbomer, Acrylates/C10-30 Alkyl Acrylate Crosspolymer, and combinations thereof. In other examples, the topical skin care composition may comprise a thickening agent selected from the group consisting of carboxylic acid polymers, crosslinked polyacrylate polymers, polyacrylamide polymers, polysaccharides, gums, crosslinked vinyl ether/maleic anhydride copolymers, crosslinked poly(N-vinylpyrrolidones), and mixtures thereof. Preferably, the topical skin care composition may comprise a thickening agent selected from the group consisting of carboxylic acid polymers, crosslinked polyacrylate polymers, polyacrylamide polymers, and mixtures thereof, more preferably selected from the group consisting of crosslinked polyacrylate polymers, polyacrylamide polymers, and mixtures thereof.

### Vitamins and Derivatives Thereof

The continuous and/or the dispersed phase can include a safe and effective amount of one or more vitamins and derivatives thereof. Nonlimiting examples of vitamins and derivatives thereof may include B3 compounds such as niacinamide, niacinnicotinic acid, tocopheryl nicotinate; B5 compounds, such as panthenol; vitamin A compounds and natural and/or synthetic analogs of Vitamin A, including retinoids, retinol, retinyl acetate, retinyl palmitate, retinoic acid, retinaldehyde, retinyl propionate, carotenoids (pro-vitamin A); vitamin E compounds, or tocopherol, including tocopheryl sorbate, tocopheryl acetate; vitamin C compounds, including ascorbate, ascorbyl esters of fatty acids, and ascorbic acid derivatives such as magnesium ascorbyl phosphate and sodium ascorbyl phosphate, ascorbyl glucoside, and ascorbyl sorbate), and derivatives of any of the aforementioned actives. The term “derivative” as used herein refers to structures which are not shown but which one skilled in the art would understand are variations of the basic compound.

### Thickening Agents

The continuous and/or the dispersed phase can include one or more thickening agents. The thickening agent can include natural gums, cellulose thickening agents, or a combination thereof.

Natural gums are polysaccharides of natural origin, capable of causing a viscosity increase in solution, even at small concentrations. They can be used as thickening agents, gelling agents, stabilizers, and can help keep the emulsion more stable. Most often these gums are found in the woody elements of plants or in seed coatings. Natural gums can be classified according to their origin. They can also be classified as uncharged or ionic polymers (polyelectrolytes), examples of which include the following: Natural gums obtained from seaweeds, such as: agar; alginic acid; sodium alginate; and carrageenan. Natural gums obtained from non-marine botanical resources include: gum arabic, from the sap of Acacia trees; gum ghatti, from the sap of Anogeissus trees; gum tragacanth, from the sap of Astragalus shrubs; karaya gum, from the sap of Sterculia trees. Examples of uncharged gums include: guar gum, from guar beans, locust bean gum, from the seeds of the carob tree; beta-glucan, from oat or barley bran; chicle gum, an older base for chewing gum obtained from the chicle tree; dammar gum, from the sap of Dipterocarpaceae trees; glucomannan from the konjac plant; mastic gum, a chewing gum from ancient Greece obtained from the mastic tree; psyllium seed husks, from the Plantago plant; spruce gum, a chewing gum of American Indians obtained from spruce trees; tara gum, from the seeds of the tara tree. Natural gums produced by bacterial fermentation include gellan gum and xanthan gum.

Examples of cellulose thickening agents which may be used in the skin care compositions can include, but are not limited to, hydroxyethylcellulose, hydroxypropyl cellulose, hydroxypropyl methylcellulose, microcrystalline cellulose, or a combination thereof.

### Feel Modifying Particles

The continuous and/or the dispersed phase can include one or more feel modifying particles. The topical skin care composition may include 0.001% to 25% (e.g., 0.01% to 20%, 0.05% to 15%, 0.1% to 10%, or even 0.2% to 5%) by weight of a feel modifying particle. For example, the composition may include spherical or non-spherical particles that have a mean particle size of less than 125 m (e.g., less than 100 m, 75 m, 50 m, 40 m, 30 m, 20 m, or even less than 15 m). In some instances, it may be desirable to select a particle size of between 2 m and 40 m (e.g., 10 to 25 m). If the particles are too big, they may feel abrasive when the composition is applied to sensitive skin. Particle size can be determined by any suitable method known in the art, such as by using Coulter counter equipment, laser diffraction equipment (e.g., LA-960 from Horiba Scientific or the like), dynamic or static image analysis equipment (e.g., Camsizer® from Horiba Scientific or the like), or optical microscopy (e.g., ASTM designation E20-85 titled “Standard Practice for Particle Size Analysis of Particulate Substances in the Range of 0.2 to 75 Micrometers by Optical Microscopy” ASTM Volume 14.02, 1993).

Some non-limiting examples of particles that may be suitable include polymeric particles chosen from the methylsilsesquioxane resin microspheres (e.g., the TOSPEARL® series of spherical silicone resin beads from Momentive Performance Materials, Inc.); microspheres of polymethylmethacrylates (Micropearl™ M 100 from Seppic); spherical silicone elastomer particles of crosslinked polydimethylsiloxanes (e.g., KSP-100, KSP-101, KSP-102, KSP-103, KSP-104, and KSP-105 all from Shin Etsu); spherical particles of polyamide (e.g., nylon-12 and Orgasol™ 2002D Nat C05 from Atochem); polystyrene microspheres (e.g., (Dynospheres™ from ThermoFisher Scientific); ethylene acrylate copolymer (e.g., FloBead™ EA209 from Kobo); aluminum starch octenylsuccinate (e.g., the Dry Flo™ series of coated and uncoated spherical starch particles from Akzo Nobel); microspheres of polyethylene (e.g., Microthene™ FN510-00 from Equistar), and combinations of these.

Another example of a material that may provide suitable feel properties is a silicone elastomer. A silicone elastomer can help reduce the tackiness of the composition (e.g., caused by non-volatile oils) and provide a more lubricious feel upon application. Sone non-limiting examples of silicone elastomers are crosslinked organopolysiloxane (or siloxane) elastomers, as described in U.S. Patent Publication No. 2003/0049212. The elastomers may be emulsifying or non-emulsifying silicone elastomers. “Emulsifying,” as used herein, means crosslinked organopolysiloxane elastomers having at least one polyoxyalkylene 30 (e.g., polyoxyethylene or polyoxypropylene) or polyglycerin moiety, whereas “non-emulsifying” means crosslinked organopolysiloxane elastomers essentially free of polyoxyalkylene or polyglycerin moieties. Some non-limiting examples of silicone elastomers that may be suitable for use herein are disclosed in U.S. Publication Nos. 2013/0243835, 2003/0049212, and 2002/0022040; and U.S. Pat. Nos. 5,412,004; 5,837,793; and 5,811,487.

Still other examples of materials that may provide desirable feel properties include low-melting-point waxes (e.g., hydrocarbon waxes and/or silicone waxes). A low-melting-point wax may be selected to impart a dry feel when touched initially and a lubricious feel in use (i.e., upon melting). The low-melting-point wax, upon melting, may also facilitate the ease of spreading, rubbing or otherwise applying skin care composition. Some non-limiting examples of silicone waxes that may be suitable for use herein are described in U.S. Publication No. 2004/0197286. The melting point of the low-melting-point waxes herein is selected to be less than the average human body temperature, which is typically about 37° C. For example, the low-melting-point wax may have a melting point of between 25° C. and 37° C. (e.g., 28° C. to 35° C.), which can be determined using the drop melting point described in ASTM D127.

Still other examples of materials that may provide desirable feel properties are starches. Starches may be sourced from corn, wheat, potato, rice, cassava and tapioca. Starches may be unmodified, modified, or partially degraded. Modified starch may include cationic starch, hydroxyethyl starch, carboxymethylated starch, and polylactic acid graft-starch and polycaprylactone graft starch. Degraded starches may include dextrin and maltodextrin preferably with a dextrose equivalent of 30 or lower. Preferably the starch is a corn starch and/or a modified corn starch.

Still other examples of materials that may provide desirable feel properties are natural powders and can include, for example, clay, diatomaceous earth, fuller's earth, silica, silica shells or spherical silica, fumed silica, spherical silica, hydrated silica, silica silylate, mica, titanated mica, talc, corn starch, rice starch, glyceryl starch, soy flour, walnut shell powder, agar, sericite, dextran, nylon, silk powder, chalk, calcium carbonate, bismuth oxychloride, iron oxide, titanium dioxide, aluminum silicate, magnesium aluminum silicate, calcium silicate, magnesium trisilicate, aluminum starch octenylsuccinate, bentonite, hectorite, kaolin, maltodextrin, montmorillonite, zinc laurate, zinc myristate, zinc rosinate, alumina, attapulgite, tin oxide, titanium hydroxide, trimagnesium phosphate, or a mixture thereof.

The feel modifying particles may include colloidal oatmeal. The topical skin care composition may comprise from about 0.001% to about 25%, or from about 0.007% to about 20%, preferably from about 0.5% to about 10%, more preferably from about 1% to about 5% or from about 1% to about 3% by weight of the composition of colloidal oatmeal.

Colloidal oatmeal, which is made by grinding and/or processing oat grain into a powder, is known for use in the cosmetics and pharmaceutical industries as a skin protectant. To produce colloidal oatmeal, the oat grain may be ground and/or processed until no more than 3% of the total particles in the powder exceed 150 m in size and no more than 20% exceed 75 m in size. The small size of the particles may contribute to their ability to deposit on the skin and form an occlusive barrier when dispersed in water. For example, colloidal oatmeal has been used for bar soaps, bath powders, lotions, and poultices to treat skin that has been damaged, irritated, or distressed by a wide variety of causes. Colloidal oatmeal may be from any oat species (i.e., Avena abyssinica, Avena byzantine, Avena nuda, Avena strigose, Avena, sativa).

### Other Optional Ingredients

The continuous and/or the dispersed phase can include one or more additional ingredients commonly used in cosmetic compositions (e.g., dyes, pigments, mica, insoluble particles, skin care actives, anti-inflammatory agents, sunscreen agents, emulsifiers, buffers, rheology modifiers, chelants, combinations of these and the like), provided that the additional ingredients do not undesirably alter the skin health or appearance benefits provided by the present compositions. The additional ingredients can be in the continuous phase and/or the dispersed phase. The additional ingredients, when incorporated into the composition, should be suitable for use in contact with human skin tissue without undue toxicity, incompatibility, instability, allergic response, and the like. Some nonlimiting examples of additional actives include vitamins, minerals, peptides and peptide derivatives, sugar amines, sunscreens, oil control agents, particulates, flavonoid compounds, hair growth regulators, anti-oxidants and/or anti-oxidant precursors, preservatives, protease inhibitors, tyrosinase inhibitors, anti-inflammatory agents, moisturizing agents, exfoliating agents, skin lightening agents, sunless tanning agents, lubricants, anti-acne actives, anti-cellulite actives, chelating agents, anti-wrinkle actives, anti-atrophy actives, phytosterols, botanical extracts, essential oils derived from plants and/or plant hormones, N-acyl amino acid compounds, antimicrobials, and antifungals. In some examples, the composition can include a fragrance, in particular a natural fragrance or an essential oil imparting an aroma, or a colorant, in particular a natural colorant. In some examples, the skin care composition can include a silicone elastomer including but not limited to silicone elastomers supplied solvated in non-volatile linear silicone fluids, silicone elastomers supplied solvated in alkanes, esters or triglycerides. The chelant can be selected from ethylenediaminetetraacetic acid (EDTA), trisodium ethylenediamine disuccinate (EDDS), trisodium dicarboxymethyl alaninate, tetrasodium glutamate diacetate, phytic acid, or a combination thereof. Other non-limiting examples of additional ingredients and/or skin care actives that may be suitable for use herein are described in U.S. Publication Nos. 2002/0022040; 2003/0049212; 2004/0175347; 2006/0275237; 2007/0196344; 2008/0181956; 2008/0206373; 2010/00092408; 2008/0206373; 2010/0239510; 2010/0189669; 2010/0272667; 2011/0262025; 2011/0097286; US2012/0197016; 2012/0128683; 2012/0148515; 2012/0156146; and 2013/0022557; and U.S. Pat. Nos. 5,939,082; 5,872,112; 6,492,326; 6,696,049; 6,524,598; 5,972,359; and 6,174,533.

The optional ingredients can include natural or plant extracts include extracts obtained from the following plants: Centella asiatica; Phytolacca decandra, Conium maculatum, Rhus toxicodendron, Sanguinaria canadensis, Berberis vulgaris, Matricaria chamomilla, Achillea millefolium, Aesculus hippocastanum, Hamamelis Virginiana, Populus alba, Populus Tremuloides, Cochlearia Officinalis, Lens Esculenta, Ervum lens, Scrophularia Nodosa, Veronica Officinalis, Althaea Officinalis, Sambucus Nigra, Malva Sylvestris, Nasturtium Officinale, Smilax Medica, Tussilago Farfara, Pimpinella Saxifraga, Vincetoxicum Hirundinaria, Avena sativa.

The optional ingredient may be a sensate material. Sensates provide a sensory benefit, such as a warming, tingling, or cooling sensation. The sensate can be chosen from 3-1-menthoxypropane-1,2-diol, menthyl lactate; N,2,3-trimethyl-2-isopropylibutanamide; N-ethyl-p-menthan-3-carboxamide: N-(4-cyanomethylphenyl)-p-menthanearboxamide, and combinations thereof, more preferably, 3-1-menthoxypropane-1,2-diol, menthyl lactate; N,2,3-trimethyl-2-isopropylbutanamide; N-ethyl-p-menthan-3-carboxamide; N-(4-cyanomethylphenyl)-p-menthanecarboxamide, or mixtures thereof. In an example, the sensate can be menthyl lactate.

When including optional ingredients in the compositions herein, it may be desirable to select ingredients that do not form complexes or otherwise undesirably interact with other ingredients in the composition, especially pH sensitive ingredients like niacinamide, salicylates and peptides. When present, the optional ingredients may be included at amounts of from 0.0001% to 50%; from 0.001% to 20%; or even from 0.01% to 10% (e.g., 50%, 40%, 30%, 20%, 10%, 5%, 4%, 3%, 2%, 1%, 0.5% or 0.1%), by weight of the composition. Alternatively, the optional ingredients may be included at amounts of from about 0.000001% to about 0.02%, preferably about 0.00001% to about 0.01%, or from about 0.00001% to about 0.001%, more preferably 0.0001% to about 0.001%.

### Methods of Using Topical Skin Care Compositions

The topical skin care compositions may be used to soothe irritated skin; relieve itchy, dry, irritated skin; and/or moisturize to soften skin using such topical skin care compositions. The skin care composition can heal, relieve, soothe, and/or repair dry, cracked skin. The skin care composition can create a protective barrier that can lock in moisture and keep your skin hydrated. The skin care composition can provide results that are noticeable to a user within days, weeks, and/or one or more months.

The topical skin care compositions may be used to treat, relieve, and/or soothe symptoms of psoriasis, eczema, xerosis that can include itch, redness, flaking, cracking, scaling, tightness, and/or roughness of the skin, scalp, and/or mucous membrane, and/or otherwise dry skin and/or irritated skin. The treatment method may include the steps of providing a topical skin care composition according to the present disclosure and applying a safe and effective amount of the topical skin care composition to at least a portion of a user's skin, scalp, and/or mucous membrane in need thereof.

The itch, redness, flaking, cracking, scaling, tightness, and/or roughness may be associated with at least one skin, scalp, and/or mucous membrane condition selected from eczematous dermatitis, contact dermatitis, atopic dermatitis, psoriasis and/or xerosis. Other conditions wherein these symptoms may be experienced are also contemplated. The topical skin care composition may also be applied to the skin in need thereof, including but not limited to the affected situs, the surrounding skin, and generally the skin all over the body, when there are active flares present as well as when there are no active flares present. The portion of the user's skin, scalp, and/or mucous membrane in need thereof may be presently affected by one or more of the following: rash, scaling, lesions, fissures, and/or bumps, or, none of the above may be present. The term “skin, scalp, and/or mucous membrane in need thereof”, is meant to be construed as narrowly as the affected situs where itch, redness, flaking, dryness, and/or roughness is present and/or where an active flare is present to as broadly as the skin of the body as a whole. The topical skin care composition may provide the benefit of relief, preferably fast relief, of itch, redness, flaking, dryness, and/or roughness as well as other bothersome symptoms associated with eczematous dermatitis, contact dermatitis, atopic dermatitis, and/or psoriasis during active flares. By “relief”, it is meant that the user may feel relief or improvement of the symptoms after applying the topical skin care composition. By “fast relief”, it is meant that the user may feel relief or improvement of the symptoms immediately after applying the topical skin care composition, within one about (1) day after applying the topical skin care composition, within about four (4) days after applying the topical skin care composition, within about one (1) week after applying the topical skin care composition, within about two (2) weeks after applying the topical skin care composition, within about four (4) weeks after applying the topical skin care composition, within about eight (8) weeks after applying the topical skin care composition, and so on, generally less than about twelve (12) weeks after applying the topical skin care composition. Preferably, the topical skin care composition may provide the benefit of fast relief of itch, redness, flaking, dryness, and/or roughness as well as other bothersome symptoms associated with eczematous dermatitis, contact dermatitis, atopic dermatitis, xerosis, and/or psoriasis during active flares within one (1) to seven (7) days after applying the topical skin care application.

The method may include the step of providing a topical skin care composition according to the present disclosure. The topical skin care composition may be in any product form that enables a user to apply the composition to the user's skin, scalp, and/or mucous membrane. Examples include, but are not limited to, the product forms as disclosed herein. The topical skin care composition may be provided in a vessel depending upon the product form, such as for example, in ajar, in a squeeze tube, in a bottle with a pump dispenser, in a canister, or any such vessel known to one skilled in the art to deliver topical skin care compositions.

Many regimens exist for the application of topical skin care compositions. The step of applying a safe and effective amount of the topical skin care composition to at least a portion of a user's skin, scalp, and/or mucous membrane in need thereof may be performed, by way of nonlimiting example, as follows. A user may obtain the provided topical skin care composition and apply a liberal amount of the topical skin care composition to the skin, scalp, and/or mucous membrane in need thereof, preferably with even more liberal application any affected situses and area surrounding the affected situses. The step of applying the topical skin care composition to the portion of the user's skin, scalp, and/or mucous membrane in need thereof may comprise applying from about 0.01 g/m² to about 20 g/m², or from about 0.05 g/m² to 15 g/m², or from about 0.1 g/m² to about 10 g/m² of the topical skin care composition.

In a nonlimiting example, the step of applying the topical skin care composition to the portion of the user's skin, scalp, and/or mucous membrane in need thereof is repeated at least once, preferably at least twice, or on a more frequent basis, within a twenty-four-hour time period. When applied twice daily, the first and second applications may be separated by at least 1 to 12 hours. Typically, the topical skin care composition may be applied in the morning and/or in the evening before bed.

The step of applying the topical skin care composition may be repeated during a treatment period. The treatment period is ideally of sufficient time to provide a relief or improvement of their symptoms and to also improve skin health. The treatment period may be up until the user experiences relief or improvement in their symptoms, but preferably the treatment period continues even after the user experiences relief or improvement their symptoms so that the topical skin care composition may gradually improve the health of the skin. Long-term application of the topical skin care composition after the user experiences relief or improvement of challenged skin symptoms may potentially lessen the frequency and/or severity of future occurrences of challenged skin symptoms. The treatment period may be at least 2 consecutive days, at least 3 consecutive days, at least 4 consecutive days, at least 5 consecutive days, at least 6 consecutive days, at least 1 week, at least 2 weeks, at least 3 weeks, at least 4 weeks, at least 5 weeks, at least 6 weeks, at least 7 weeks, at least 8 weeks, and so on. The treatment period may extend over multiple months (i.e., 3-12 months) or multiple years. In a nonlimiting example, the topical skin care composition may be applied at least once, preferably at least twice, a day during a treatment period of at least 1 week, about 2 weeks, about 3 weeks, or about 4 weeks. The topical skin care composition may be concurrently applied in addition to any other prescription and/or non-prescription compositions the user may be taking.

In a nonlimiting example, after application of the topical skin care composition to the user's skin, scalp, and/or mucous membrane in need thereof, the topical skin care composition is not removed for at least five minutes. Applicant has found that even leaving the topical skin care composition without removal for at least five minutes may provide some relief or improvement of itch, redness, flaking, dryness, and/or roughness of the skin, scalp, and/or mucous membrane. Preferably, the topical skin care composition is not removed for at least thirty minutes, more preferably for at least one hour, even more preferably for at least several hours, or until the next application of the topical skin care composition. In applications such as when the topical skin care composition is used for the scalp as a rinse-off product, it is particularly beneficial for the topical skin care composition to not be removed for at least five minutes. A user may cleanse the skin, scalp, and/or mucous membrane prior to application of the topical skin care composition such as by using a bar of soap or other conventional cleansers. A user may apply the topical skin care composition without cleansing the skin, scalp, and/or mucous membrane prior to application. After applying the topical skin care composition, a user may occlude the topical skin care composition with, for example, a bandage. Occluding with a bandage may keep the topical skin care composition from being rubbed off of the skin, scalp, and/or mucous membrane by external objects, allowing for longer exposure of the skin, scalp, and/or mucous membrane to the composition.

### Test Methods

### Rheology Test Method

This method provides a suitable means of measuring the viscosity of the compositions herein. The instrument used in this method is a TA Instruments brand rheometer, or equivalent. The instrument is set up to conduct a rotational ramp under controlled stress conditions, from 0.01 to 2000 Pa over 240 seconds, using serrated parallel plate for the upper and lower geometry with a 40 mm diameter, and a 1.0 mm gap. The temperature is set to 25° C. The instrument protocol is set up to collect 100 data points per decade in a logarithmic distribution, with a setting to stop the measure when shear rates exceed 110 s⁻¹.

After conducting an automated gap calibration, approximately 5 grams of a sample is placed on the center of the plate. The plate is positioned to provide a gap of 1.05 mm, and any excess material squeezed out of the perimeter gap of the parallel plates is carefully trimmed away. The plate is then moved the final 0.05 mm to provide a 1.00 mm gap, and the measurement is initiated. At the end of the run, the data file is saved for subsequent plotting and analysis. The viscosity is reported in units of Pascal-Seconds (PaS) as the value at or near a shear rate of 0.10 s⁻¹.

### Brookfield Viscosity Test Method

The Brookfield viscosity of composition is determined using a BROOKFIELD® DV2 rotational viscometer, at 5 rpm, at 20-21° C. and using Helipath with Spindle TC 93.

### Tack Method

This method provides a suitable means for determining the tackiness of the compositions described herein. The method uses a texture analyzer to contact a probe with a film formed from the composition. The texture analyzer then measures the force needed to separate the probe from the composition film. Tack Force can be determined by this method. The Tack Method is configured to run for a period of 100 minutes. It is believed, without being limited by theory, that the tackiness properties exhibited by a composition over the course of the Tack Test approximate the tackiness properties exhibited by the composition during the first few minutes (e.g., less than 10 minutes, between 30 seconds and 5 minutes, or between 1 minute and 3 minutes) of use of the composition by a user.

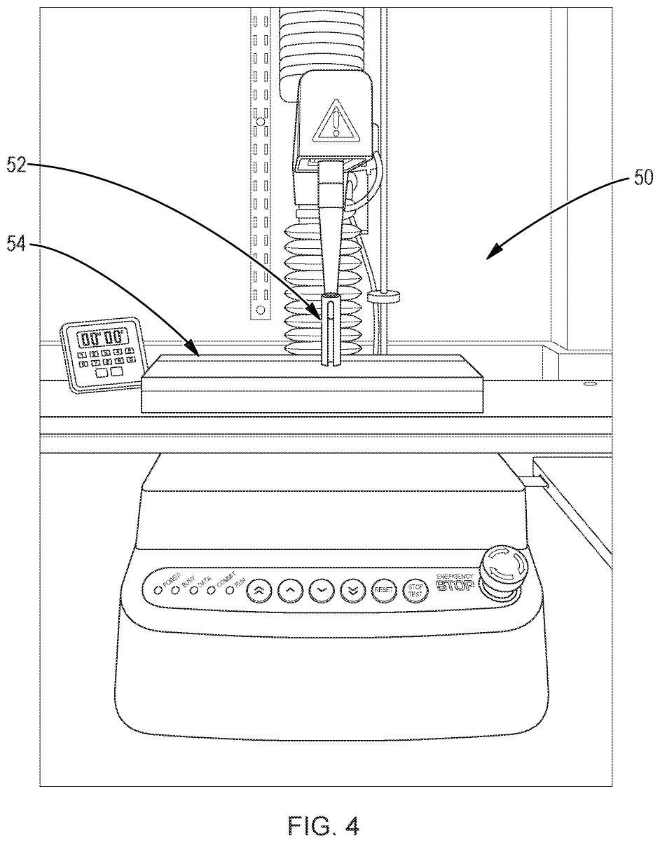

FIG. 4 illustrates and exemplary test setup for the Tack Test. A TA.XT2i brand texture analyzer (available from Texture Technologies Corporation, MA) 50 or equivalent is used to measure the tack of a film formed from a composition of interest. The texture analyzer is equipped with a tack probe 52 in the form of a 12.75 mm diameter acrylic cylinder with a flat surface. During testing, the flat surface of the probe 52 is contacted with the surface of a film of the composition. Thus, the flat surface of the probe and the surface of the film must be parallel to one another during testing to ensure there is sufficient contact by the film across the effective test surface of the probe 52. The film is made by filling or overfilling (followed by draw down) a rectangular channel 54 (e.g., 25 cm long×30 mm wide a 0.25 mm deep) with the composition to be tested.

The test is conducted using an adhesive test protocol with a pretest speed of 0.10 mm/second, a test speed of 0.10 mm/sec and a post-test speed of 1.0 mm/sec. The applied force is 200 g, the return distance is 4 mm and the contact time is 5.0 sec. The trigger type to designate sample contact is set to automatic and the trigger force is 5.0 g. The test is run and at the following time increments immediately after the film is prepared: <1 minute (i.e., immediately following preparation of the film), 10, 20, 30, 40, 50, 60, 80, and 100 minutes. Each time point is run on a previously undisturbed/untested area of the sample. Each sample is run in triplicate and the averages are recorded.

The average tack force between 20 and 40 minutes can be useful in determining whether a product is too tacky to be consumer acceptable. To calculate the average tack force between 20 and 40 minutes, the tack force at 20, 30, and 40 minutes are added together and divided by three and is reported in units of grams (g).

### % Transmittance Test Method

Clarity can be measured by % Transmittance (% T) using Ultra-Violet/Visible (UV/VI) spectrophotometry which determines the transmission of UV/VIS light through a sample. A light wavelength of 600 nm has been shown to be adequate for characterizing the degree of light transmittance through a sample. Typically, it is best to follow the specific instructions relating to the specific spectrophotometer being used. In general, the procedure for measuring percent transmittance starts by setting the spectrophotometer to 600 nm. Then a calibration “blank” is run to calibrate the readout to 100 percent transmittance. A single test sample is then placed in a cuvette designed to fit the specific spectrophotometer and care is taken to ensure no air bubbles are within the sample before the % T is measured by the spectrophotometer at 600 nm. Alternatively, multiple samples can be measured simultaneously by using a spectrophotometer such as the SpectraMax M-5 available from Molecular Devices. Multiple samples are transferred into a 96 well visible flat bottom plate (Greiner part #655-001), ensuring that no air bubbles are within the samples. The flat bottom plate is placed within the SpectraMax M-5 and % T measured using the Software Pro v.5™ software available from Molecular Devices.

### EXAMPLES

The following data and examples are provided to help illustrate the skincare compositions described herein. The exemplified compositions are given solely for the purpose of illustration and are not to be construed as limitations of the present disclosure, as many variations thereof are possible without departing from the spirit and scope of the disclosure. All parts, percentages, and ratios herein are by weight unless otherwise specified.

The Examples in Table 1 to Table 3 and Table 6 below, were prepared by the following method. First, a water phase was made by combining water and water-soluble ingredients including, but not limited to, glycerin, chelant (e.g., disodium EDTA, EDDS), water soluble skin care actives (e.g., panthenol), and antioxidants (e.g., hydroxyacetophenone). The water phase was mixed and heated to at least about 50° C. but less than about 80° C. until the ingredients were in solution and/or dispersed. The water phase can contain about 70% to about 98%, alternatively from about 75% to about 95%, alternatively from about 80% to about 94%, alternatively from about 85% to about 93%, or alternatively from about 88% to about 92% water, by weight of the water phase.

In a second container, an oil phase premix was made. The premix included the liquid and waxy emollients and one or more emulsifiers. The premix was mixed and heated to at least about 50° C. but less than about 80° C. until the ingredients are fully melted or dispersed in the premix.

The premix was added to the water phase. Then, the mixture was milled to emulsify the oil phase premix into the water phase, forming an emulsion where the water phase is the continuous phase, and the oil phase is dispersed phase. One or more additional ingredients were added after emulsification while continuously mixing with an overhead mixer. Additional ingredients may be added either before, after or during cooling of the product to room temperature and included but were not limited to polymeric thickeners (e.g., SEPILIFE™ NUDE, NATURSOL™ EMI 132), silicones, colorants, pigments, powders, micas, preservatives, fragrances, and additional skin actives.

The viscosity of the Examples in the following Tables was determined using the Rheology Test Method, as disclosed herein.

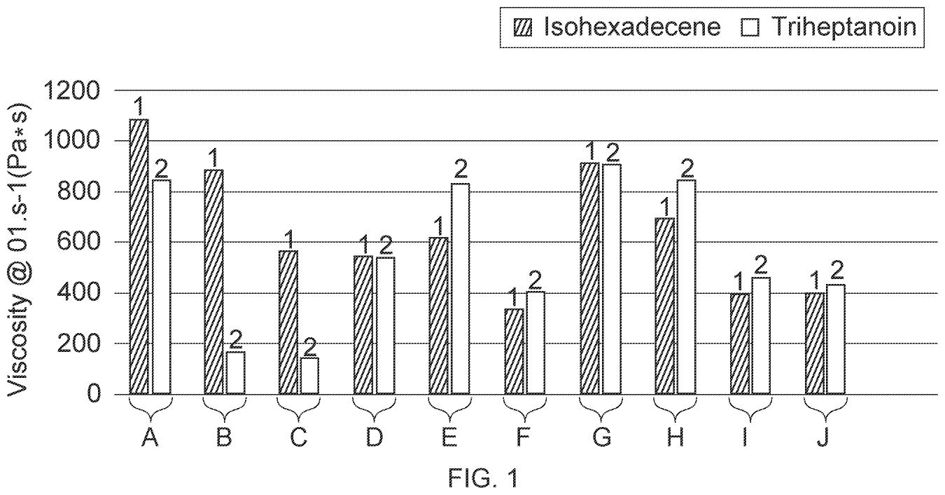

For the Examples detailed in Table 1 to Table 3, below, and FIG. 1, various emulsifiers were tested in skin care compositions. Each emulsifier was tested in a composition with an emollient that included either isohexadecene, a non-polar hydrocarbon oil, or triheptanoin, a triglyceride that is a more polar, as compared to isohexadecene. The emulsifier may be consumer acceptable if it helped build sufficient viscosity, as indicated when the composition had a viscosity greater than or equal to 550 Pa*s, according to the Rheology Test Method, described herein. This viscosity is considered the minimum necessary to create an acceptable moisturizer composition, compositions with higher viscosities may be more consumer preferred and in some examples the viscosity can be increased by adding more emulsifier and/or polymer. It can be preferred to select an emulsifier that yields a composition with sufficient viscosity with both isohexadecene and tripheptanoin emollients because this shows that the emulsifier builds viscosity across a range of products with different emollient polarities. Also, many skin care products use more than one emollient with different polarities to aid in solubility of various skin care benefit ingredients including ingredients that improve skin texture, tone, or protect against UV damage.

Some examples were made with a feel modification particle wherein the feel modification particle was either a modified corn starch or Zea mays corn starch (commercially available as AMAZE® HTP Pure from NOURYON®). It was found that there was no appreciable difference in viscosity and stability when formulating with either starch.

TABLE 1

| | Ex. | Ex. | Ex. | Ex. | Ex. | Ex. | Ex. | Ex. |
|---|---|---|---|---|---|---|---|---|
| | A-1 | A-2 | B-1 | B-2 | C-1 | C-2 | D-1 | D-2 |
| | (wt. | (wt. | (wt. | (wt. | (wt. | (wt. | (wt. | (wt. |
| | %) | %) | % | %) | %) | %) | %) | %) |
| Sodium Acrylate/Sodium | 1.6 | 1.6 | 1.6 | 1.6 | 1.6 | 1.6 | 1.6 | 1.6 |
| Acryloyldimethyl Taurate | | | | | | | | |
| Copolymer & C15-19 | | | | | | | | |
| Alkane & Polyglyceryl-6 | | | | | | | | |
| Laurate & Polyglycerin-6¹ | | | | | | | | |
| Panthenol | 1 | 1 | 1 | 1 | 1 | 1 | 1 | 1 |
| Glycerin | 15 | 15 | 15 | 15 | 15 | 15 | 15 | 15 |
| Niacinamide | 5 | 5 | 5 | 5 | 5 | 5 | 5 | 5 |
| Ethylenediaminetetraacetic | 0.05 | 0.05 | 0.05 | 0.05 | 0.05 | 0.05 | 0.05 | 0.05 |
| acid (EDTA) | | | | | | | | |
| Hydroxyacetophenone | 0.15 | 0.15 | 0.15 | 0.15 | 0.15 | 0.15 | 0.15 | 0.15 |
| Modified Corn Starch | | | 5 | 5 | 5 | 5 | 5 | 5 |
| Zea Mays Corn Starch² | 5 | 5 | | | | | | |
| Phenoxyethanol | 0.375 | 0.375 | 0.375 | 0.375 | 0.375 | 0.375 | 0.375 | 0.375 |
| Ethylhexylglycerin | 0.2 | 0.2 | 0.2 | 0.2 | 0.2 | 0.2 | 0.2 | 0.2 |
| Isohexadecene | 6 | | 6 | | 6 | | 6 | |
| Triheptanoin | | 6 | | 6 | | 6 | | 6 |
| Isoamyl Laurate³ | 3 | 3 | 3 | 3 | 3 | 3 | 3 | 3 |
| Stearyl Alcohol | 1 | 1 | 1 | 1 | 1 | 1 | 1 | 1 |
| Cetyl Alcohol | 0.8 | 0.8 | 0.8 | 0.8 | 0.8 | 0.8 | 0.8 | 0.8 |
| Behenyl Alcohol | 0.6 | 0.6 | 0.6 | 0.6 | 0.6 | 0.6 | 0.6 | 0.6 |
| PEG-100 Stearate | 0.2 | 0.2 | | | | | | |
| Polyglyceryl-3 | | | 0.2 | 0.2 | | | | |
| Methylglucose Distearate⁴ | | | | | | | | |
| Polyglyceryl-10 | | | | | 0.2 | 0.2 | | |
| Diisostearate⁵ | | | | | | | | |
| Polyglyceryl-6 Distearate⁶ | | | | | | | 0.2 | 0.2 |
| Water | Q.S. to Volume | | | | | | | |
| Viscosity (Pa*s)** | 1082 | 877 | 167 | 480 | 560 | 141 | 542 | 534 |

**Measured using the Rheology Test Method

TABLE 2

| | Ex. E-1 | Ex. E-2 | Ex. F-1 | Ex. F-2 | Ex. G-1 | Ex. G-2 |
|---|---|---|---|---|---|---|
| | (wt.%) | (wt. %) | (wt. %) | (wt. %) | (wt. %) | (wt. %) |
| Sodium Acrylate/Sodium | 1.6 | 1.6 | 1.6 | 1.6 | 1.6 | 1.6 |
| Acryloyldimethyl Taurate | | | | | | |
| Copolymer & C15-19 | | | | | | |
| Alkane & Polyglyceryl-6 | | | | | | |
| Laurate & Polyglycerin-6¹ | | | | | | |
| Panthenol | 1 | 1 | 1 | 1 | 1 | 1 |
| Glycerin | 15 | 15 | 15 | 15 | 15 | 15 |
| Niacinamide | 5 | 5 | 5 | 5 | 5 | 5 |
| Ethylenediaminetetraacetic | 0.05 | 0.05 | 0.05 | 0.05 | 0.05 | 0.05 |
| acid (EDTA) | | | | | | |
| Hydroxyacetophenone | 0.15 | 0.15 | 0.15 | 0.15 | 0.15 | 0.15 |
| Modified Corn Starch | 5 | 5 | 5 | | | |
| Zea Mays Corn Starch² | | | | 5 | 5 | 5 |
| Phenoxyethanol | 0.375 | 0.375 | 0.375 | 0.375 | 0.375 | 0.375 |
| Ethylhexylglycerin | 0.2 | 0.2 | 0.2 | 0.2 | 0.2 | 0.2 |
| Isohexadecene | 6 | | 6 | | 6 | |
| Triheptanoin | | 6 | | 6 | | 6 |
| Shea Butter Ethyl Esters | | | 3 | 3 | | |
| Isoamyl Laurate³ | 3 | 3 | | | 3 | 3 |
| Stearyl Alcohol | 1 | 1 | 1 | 1 | 1 | 1 |
| Cetyl Alcohol | 0.8 | 0.8 | 0.8 | 0.8 | 0.8 | 0.8 |
| Behenyl Alcohol | 0.6 | 0.6 | 0.6 | 0.6 | 0.6 | 0.6 |
| Polyglyceryl-3 Distearate | 0.2 | 0.2 | | | | |
| (and) Glyceryl Stearate | | | | | | |
| Citrate⁷ | | | | | | |
| Glyceryl Stearate⁸ | | | 0.2 | 0.2 | | |
| Polyglyceryl-3 Cetearyl | | | | | 0.2 | 0.2 |
| Ether Olivate⁹ | | | | | | |
| Water | Q.S. to Volume | | | | | |
| Viscosity (Pa*s)** | 615 | 827 | 335 | 402 | 911 | 906 |

**Measured using the Rheology Test Method

TABLE 3

| | Ex. H-1 | Ex. H-2 | Ex. I-1 | Ex. I-2 | Ex. J-1 | Ex. J-2 |
|---|---|---|---|---|---|---|
| | (wt. %) | (wt. %) | (wt. %) | (wt. %) | (wt. %) | (wt. %) |
| Sodium Acrylate/Sodium | 1.6 | 1.6 | 1.6 | 1.6 | 1.6 | 1.6 |
| Acryloyldimethyl Taurate | | | | | | |
| Copolymer & C15-19 | | | | | | |
| Alkane & Polyglyceryl-6 | | | | | | |
| Laurate & Polyglycerin-6¹ | | | | | | |
| Panthenol | 1 | 1 | 1 | 1 | 1 | 1 |
| Glycerin | 15 | 15 | 15 | 15 | 15 | 15 |
| Niacinamide | 5 | 5 | 5 | 5 | 5 | 5 |
| Ethylenediaminetetraacetic | 0.05 | 0.05 | 0.05 | 0.05 | 0.05 | 0.05 |
| acid (EDTA) | | | | | | |
| Hydroxyacetophenone | 0.15 | 0.15 | 0.15 | 0.15 | 0.15 | 0.15 |
| Modified Corn Starch | | | | | | |
| Zea Mays Corn Starch² | 5 | 5 | 5 | 5 | 5 | 5 |
| Phenoxyethanol | 0.375 | 0.375 | 0.375 | 0.375 | 0.375 | 0.375 |
| Ethylhexylglycerin | 0.2 | 0.2 | 0.2 | 0.2 | 0.2 | 0.2 |
| Isohexadecene | 6 | | 6 | | 6 | |
| Triheptanoin | | 6 | | 6 | | 6 |
| Shea Butter Ethyl Esters | | | 3 | 3 | | |
| Isoamyl Laurate³ | 3 | 3 | | | 3 | 3 |
| Stearyl Alcohol | 1 | 1 | 1 | 1 | 1 | 1 |
| Cetyl Alcohol | 0.8 | 0.8 | 0.8 | 0.8 | 0.8 | 0.8 |
| Behenyl Alcohol | 0.6 | 0.6 | 0.6 | 0.6 | 0.6 | 0.6 |
| Cetearyl Olivate, Sorbitan | 0.2 | 0.2 | | | | |
| Olivate¹⁰ | | | | | | |
| Sucrose Polystearate (and) | | | 0.2 | 0.2 | | |
| Cetearyl Alcohol (and) Olea | | | | | | |
| Europaea (Olive) Oil | | | | | | |
| Unsaponifiables¹¹ | | | | | | |
| Glyceryl Oleate (and) | | | | | 0.2 | 0.2 |
| Polyglyceryl-3- | | | | | | |
| polyricinoleate (and) Olea | | | | | | |
| Europaea (Olive) Oil | | | | | | |
| Unsaponifiables¹² | | | | | | |
| Water | Q.S. to Volume | | | | | |
| Viscosity (Pa*s)** | 692 | 839 | 393 | 454 | 398 | 431 |

**Measured using the Rheology Test Method
Tradenames and Suppliers for Table 1 to Table 3:
¹SEPILIFE™ NUDE from SEPPIC® Corporation
*Plant based and renewable
²AMAZE® HTP Pure from NOURYON®
³MACKADERM® LIA MB from Solvay
4. TEGO® Care 450MB from Evonik
5. Barfix H from BARNET® Products
6. PLUROL® Stearique MB from GATTEFOSSÉ®
7. DERMOFEEL® NC MB from EVONIK®
8. HALLSTAR® GMS Pure from HALLSTAR®
9. GALOLIVE® Bioten from ACME-HARDESTY®
10OLIVEM® 1000 from HALLSTAR®
¹¹PLANTASENS® Natural Emulsifier HP10 from CLARIANT®
¹²PLANTASENS® Emulsifier CP5 from CLARIANT®

The emulsifier used in Examples A-1 and 2 includes PEG-100 stearate emulsifier, a polyethylene glycol ester of stearic acid. These compositions had consumer acceptable viscosity. However, some consumers prefer compositions that are free from PEG.

Examples B-J include emulsifiers that are free of PEG. In addition, the emulsifiers in Examples B-J are natural or naturally derived, and biodegradable. The examples that had a viscosity greater than or equal to 550 Pa*s for both emollients were Examples E, G, and H. Examples E-1 and 2 are not preferred because both examples had a noticeable foul odor that would be offensive to many consumers.

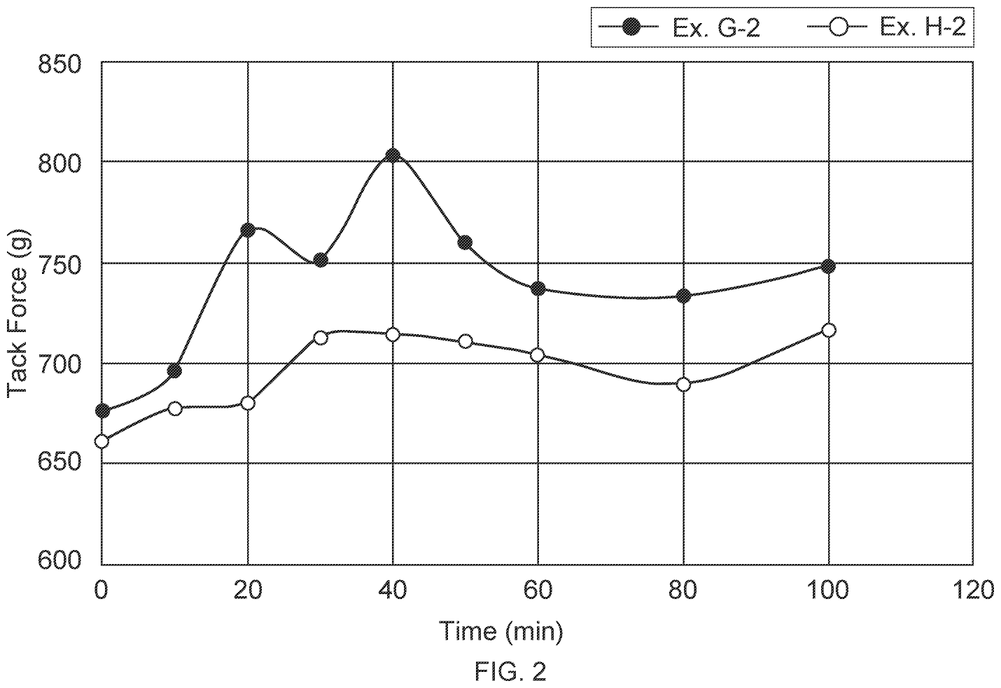

Example G-1 may be acceptable to consumers. However, Example G-2 was noticeably tacky, which can make the product difficult to spread across a user's skin and the product can feel heavy and sticky after application or could be painful if applied to dry or damaged skin. Since this emulsifier does not produce a consumer preferred product across a range of emollients, it may not be preferred (see FIG. 2, Table 4 and accompanying text for additional data and discussion regarding the tackiness of Example G-2).

Examples H-1 and 2 both have a viscosity greater than or equal to 550 Pa*s and have no noticeable off-putting smell. In addition, both examples were stable emulsions that had a smooth, silky texture. The emulsifier in Examples H-1 and 2 was cetearyl olivate. It is believed that other emulsifiers that include a mixture of waxy esters made from fatty alcohols and fatty acids derived from natural sources (e.g., olive, palm, corn, palm, rapeseed, soybean, sunflower, coconut, tallow, canola, castor) with a carbon chain length ranging from C12-C22 could be used in stable skin care compositions that have sufficient viscosity (e.g., >550 Pa*s).

FIG. 2 and Table 4, show the Tack Force for Examples G-2 (Table 2) and H-2 (Table 3). The tack force was determined according to the Tack Force Test Method, described herein. Both examples include the emollient triheptanoin at 6.0% and 0.20% of the respective emulsifiers. The emulsifier in Example G-2 was Polyglyceryl-3 Cetearyl Ether Olivate emulsifier (commercially available as GALOLIVE® Bioten, from ACME-HARDESTY®). FIG. 1 shows that Examples G-1 and 2 had the highest viscosity of the inventive Examples B-J, which initially indicated that GALOLIVE® Bioten could be consumer preferred. However, FIG. 2 and Table 4 show that Example G-2 has a greater tack force, as compared to Example H-2, particularly in the 20-to-40-minute time range of the tack method. The significance of the higher tack force is that Example G-2 is expected to feel sticky and stringy, especially when compared to Example H-2.

TABLE 4

| | Ex. G-2 (Table 2) | Ex. H-2 (Table 3) |
|---|---|---|
| Time (min) | Tack Force (g) | Tack Force (g) |
| 0 | 676 | 661 |
| 10 | 696 | 677 |
| 20 | 766 | 680 |
| 30 | 751 | 712 |
| 40 | 803 | 714 |
| 50 | 760 | 710 |
| 60 | 737 | 704 |
| 80 | 733 | 689 |
| 100 | 748 | 716 |

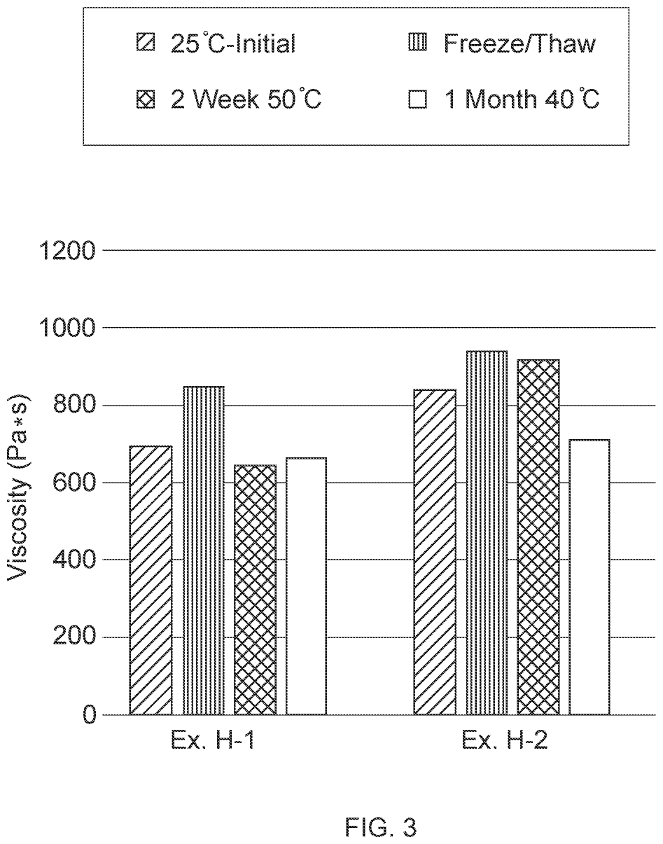

FIG. 3 shows the viscosity of Examples H-1 and H-2 (see Table 3) after it is made, after a freeze/thaw cycle, and accelerated stability conditions (i.e., after storage for 2 weeks at 50° C. and one month at 40° C.). The composition was considered stable and consumer acceptable if the viscosity varied by no more than 40% across these conditions. FIG. 3 shows that both Examples H-1 and H-2 meet the criteria for stability and are consumer acceptable.

Table 5, below, shows the results of a consumer test with consumers who self-reported moderate to very severe dry, itchy, or cracked skin. In this test, each consumer applied one of the products from Table 6 or Table 7 to their hands and body daily, as needed for four weeks. In each category, they rated the product as poor (0), fair (25), good (50), very good (75), or excellent (100), as shown in Table 6. The total score was then divided by the total number of consumers that used the product to determine the average score for each category.

From Table 5, Ex. 1 was compared to the CeraVe® Daily Moisturizing Lotion and Ex. 2 was compared to the CeraVe® Moisturizing Cream. It was found that Ex. 1 and 2 were rated higher than the corresponding CeraVe® products in the following areas: overall rating, absorbs completely, enjoyable to use, moisturized skin, reduces dryness all day, skin feels healthy, and skin looks healthy. Ex. 2 also had a statistically higher score for is not tacky/sticky. Both Ex. 1 and 2 had a score for not tacky/sticky that was above 80, which can be difficult to do in skin care compositions that provide a high degree of moisturizing because they are formulated with relatively high levels of glycerin (Ex. 1 has 7.5% and Ex. 2 has 10%) and emollients (Ex. 1 has a total emollient level of 7.5% and Ex. 2 has a total emollient level of 19%).

TABLE 5
Consumer Test

| | | Cera Ve ® | | |
|---|---|---|---|---|
| | Ex. 1 - | Daily | Ex. 3 - | Cera Ve ® |
| | Lotion | Moisturizing | Cream | Moisturizing |
| | (see | Lotion | (see | Cream |
| | Table 6) | (see Table 8) | Table 6) | (see Table 8) |
| Overall Rating | 81* | 77 | 94* | 76 |
| Absorbs completely | 85* | 79 | 90* | 76 |
| Is not tacky/sticky | 83 | 82 | 88* | 76 |
| Enjoyable to use | 76* | 70 | 86* | 72 |
| Moisturized skin | 80* | 77 | 90* | 74 |
| Reduces dryness all day | 75* | 68 | 87* | 75 |
| Skin Feels Healthy | 85* | 71 | 84* | 76 |
| Skin looks healthy | 77* | 70 | 81* | 71 |

*Statistically significant (p ≤ 0.1)

TABLE 6

| | | Ex. 1- | Ex. 2- | Ex. 3- | Ex. 4- | Ex. 5- |
|---|---|---|---|---|---|---|
| Material | Material | Lotion | Lotion | Cream | Cream | Cream |
| Function | Name | (wt. %) | (wt. %) | (wt. %) | (wt. %) | (wt. %) |
| Carrier | Purified | Q.S. | Q.S. | Q.S. | Q.S. | Q.S. |
| | Water | | | | | |
| Liquid | Dimethi- | 2.00 | 2.00 | 2.00 | 2.00 | 1.00 |
| emollient | cone | | | | | |
| Waxy | Cetyl | 1.00 | 1.50 | 2.50 | 2.50 | 2.00 |
| emollient | Alcohol | | | | | |
| Waxy | Glyceryl | 1.00 | 1.50 | 4.00 | 4.00 | 2.50 |
| emollient | Stearate | | | | | |
| Waxy | Petrolatum | 0.50 | 0.75 | 0.50 | 0.50 | 1.00 |
| emollient | | | | | | |
| Humectant | Sodium | 1.00 | 0.50 | 1.00 | 1.00 | 0.75 |
| | PCA | | | | | |
| Polymer | Carbomer¹ | 0.50 | 0.50 | 0.24 | 0.24 | 0.35 |
| Vitamin | Vitamin E | 0.10 | 0.50 | 0.10 | 0.10 | 0.20 |
| | Acetate | | | | | |
| Humectant | Glycerin | 7.50 | 8.50 | 10.00 | 7.50 | 6.50 |
| Vitamin | Niacinamide | 1.00 | 2.00 | 1.00 | 1.00 | 1.50 |
| Humectant | Propylene | 1.00 | 0.50 | 1.00 | 1.00 | 0.75 |
| | Glycol | | | | | |
| Humectant | Dihydrate | — | 1.2 | — | 3.00 | 2.00 |
| | Trehalose | | | | | |
| Preservative | Phenoxy- | 1.00 | 1.00 | 1.00 | 1.00 | 1.00 |
| | ethanol | | | | | |
| | (and) | | | | | |
| | Caprylyl | | | | | |
| | Glycol² | | | | | |
| Optional | Menthyl | 1.00 | 0.60 | 1.00 | 1.00 | 0.25 |
| ingredient | Lactate | | | | | |
| Feel | Colloidal | 1.00 | 2.00 | 1.00 | 1.00 | 1.50 |
| modifying | Oatmeal | | | | | |
| particle | | | | | | |
| Feel | Modified | 2.50 | 1.50 | — | 2.50 | — |
| modifying | Corn | | | | | |
| particle | Starch | | | | | |
| Liquid | Isopropyl | 3.00 | 2.00 | 5.00 | 2.50 | 2.50 |
| emollient | Isostearate | | | | | |
| Waxy | Shea Butter | — | — | 5.00 | 2.50 | 3.00 |
| Emollient | | | | | | |
| Emulsifier | Cetearyl | 1.00 | 0.60 | 1.00 | 1.00 | 1.30 |
| | Olivate, | | | | | |
| | Sorbitan | | | | | |
| | Olivate³ | | | | | |
| pH adjuster | Amino- | 0.27 | 0.27 | 0.27 | 0.27 | 0.27 |
| | methyl | | | | | |
| | Propanol | | | | | |
| | Brookfield | 25 | 35 | 61 | 74 | 55 |
| | Viscosity | | | | | |
| | (Pa · s)^{†} | | | | | |

^{†}Measured using the Brookfield Viscosity Method
Tradenames and Suppliers for Table 6:
¹CARBOPOL® 980 polymer
²VERSTATIL® PC
³OLIVEM® 1000

TABLE 7

| | Ingredients |
|---|---|
| CeraVe ® | aqua/water/eau, glycerin, caprylic/capric triglyceride, |
| Daily | cetearyl alcohol, cetyl alcohol, potassium phosphate, |
| Moisturizing | ceramide np, ceramide ap, ceramide eop, carbomer, |
| Lotion | dimethicone, ceteareth-20, behentrimonium methosulfate, |
| | methylparaben, sodium lauroyl lactylate, cholesterol, |
| | disodium edta, dipotassium phosphate, propylparaben, |
| | hydrolyzed hyaluronic acid, phytosphingosine, xanthan |
| | gum, polysorbate 20, polyglyceryl-3 diisostearate |
| CeraVe ® | aqua/water/eau, glycerin, cetearyl alcohol, caprylic/capric |
| Moisturizing | triglyceride, cetyl alcohol, ceteareth-20, petrolatum, |
| Cream | potassium phosphate, ceramide np, ceramide ap, ceramide |
| | eop, carbomer, dimethicone, behentrimonium methosulfate, |
| | sodium lauroyl lactylate, sodium hyaluronate, cholesterol, |
| | phenoxyethanol, disodium edta, dipotassium phosphate, |
| | tocopherol, phytosphingosine, xanthan gum, |
| | ethylhexylglycerin |

### Combinations

A. A skin care composition comprising:
   a. a continuous phase comprising:
      i. from 45 to 85% water, preferably from 55 to 85% water;
      ii. from 5% to 25%, preferably from 6% to 20%, more preferably from 4 to 18% glycerin, even more preferably from 6.5% to 18%, and even more preferably from 7.5% to 15% glycerin;
   b. a dispersed phase comprising at least two waxy emollients comprising:
      i. from 0.5 to 10% of an ester comprising from 10 to 28 carbon atoms;
      ii. from 0.1 to 5% of a fatty alcohol wherein the fatty alcohol comprises from 12 to 22;
   c. from 0.1 to 5% of a feel modifying particle chosen from siloxane, starches, colloidal oatmeal, or mixtures thereof; and
   d. 2% or less of an emulsifier comprising cetearyl olivate and sorbitan olivate.
B. The skin care composition according to Paragraph A, wherein the feel modifying particle comprises colloidal oatmeal.
C. The skin care composition according to Paragraph B, wherein the composition comprises from 0.001% to 25%, preferably from 0.007% to 20%, more preferably from 0.5% to 10%, more preferably from 1% to 5%, more preferably from 1% to 3%, and even more preferably from 0.1 to 2% of the colloidal oatmeal.
D. The skin care composition according to Paragraphs B-C, wherein the composition further comprises an additional feel modifying particle comprising modified corn starch.
E. The skin care composition according to Paragraph D, wherein the composition comprises from 0.1% to 10%, preferably from 0.5% to 8%, preferably from 1% to 5%, more preferably from 1% to 3%, and even more preferably from 1.5% to 2.5%, of the modified corn starch.
F. The skin care composition according to Paragraphs A-E, wherein the composition further comprises an additional waxy emollient chosen from petrolatum, shea butter, or mixtures thereof.
G. The skin care composition according to Paragraphs A-F, wherein the composition further comprises a liquid emollient.
H. The skin care composition according to Paragraph G, wherein the liquid emollient is chosen from dimethicone, isopropyl isostearate, or mixtures thereof.
I. The skin care composition according to Paragraphs G-H, wherein the weight ratio of waxy emollient to liquid emollient is from 1:5 to 3:1, preferably from 1:4 to 5:2, more preferably from 1:3 to 2:1, and even more preferably from 1:2 to 7:4.
J. The skin care composition according to Paragraphs G-H, wherein the weight ratio of waxy emollient to liquid emollient is greater than 1:1, preferably greater than 3:2, and more preferably greater than 5:3.
K. The skin care composition according to Paragraphs G-H, wherein the weight ratio of waxy emollient to liquid emollient is from 1:5 to 1:1, preferably from 1:4 to 4:5, and more preferably from 1:3 to 3:5.
L. The skin care composition according to Paragraphs A-K, wherein the composition comprises from 0.10% to 2% of the emulsifier, preferably from 0.2% to 1.5% of the emulsifier, more preferably from 0.4% to 1.2% of the emulsifier, and even more preferably from 0.6% to 0.8% of the emulsifier.
M. The skin care composition according to Paragraphs A-L, wherein the ester is chosen from isopropyl isostearate, glyceryl stearate, or mixtures thereof.
N. The skin care composition according to Paragraphs A-M, wherein the fatty alcohol comprises cetyl alcohol.
O. The skin care composition according to Paragraphs A-N, wherein the composition comprises from 6.5% to 10% glycerin.
P. The skin care composition according to Paragraphs A-O, comprising an average tack force between 20 and 40 minutes of less than 750 g, preferably less than 725 g, more preferably less than 710 g, and even more preferably less than 705 g according to the Tack Method described herein.
Q. The skin care composition according to Paragraphs A-P, comprising a Brookfield viscosity of from 10 Pa*s to 200 Pa*s, preferably from 12 Pa*s to 150 Pa*s, more preferably from 15 Pa*s to 125 Pa*s, and even more preferably from 20 Pa*s to 100 Pa*s according to the Brookfield Viscosity Test Method described herein.
R. The skin care composition according to Paragraphs A-Q, wherein the waxy emollients comprise cetyl alcohol, glyceryl stearate, and petrolatum.
S. The skin care composition according to Paragraphs A-R, further comprising niacinamide.
T. The skin care composition according to Paragraphs A-S, comprising a percent transparency (% T) of less than 70%, preferably less than 60%, preferably less than 50%, preferably less than 40%, preferably less than 30%, preferably less than 20%, preferably less than 10%, preferably less than 5% transmittance at 600 nm according to the % Transmittance Method.
U. Use of the skin care composition according to Paragraphs A-S to soothe irritated skin; relieve itchy, dry, and/or irritated skin; and/or moisturize to soften skin.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A skin care composition comprising:
a. a continuous phase comprising:
i. from about 45 to about 85 wt. % water;
ii. from about 4 to about 18 wt. % glycerin;
b. a dispersed phase comprising at least two waxy emollients comprising:
i. from about 0.5 to about 10 wt. % of an ester comprising from about 10 to about 28 carbon atoms;
ii. from about 0.1 to about 5 wt. % of a fatty alcohol wherein the fatty alcohol comprises from about 12 to about 22; and
c. from about 0.1 to about 5 wt. % of a feel modifying particle chosen from siloxane, starches, colloidal oatmeal, or mixtures thereof; and
d. 2 wt. % or less of an emulsifier comprising cetearyl olivate and sorbitan olivate.

2. The skin care composition of claim 1, wherein the ester is chosen from isopropyl isostearate, glyceryl stearate, or mixtures thereof and the fatty alcohol comprises cetyl alcohol.

3. The skin care composition of claim 2, wherein the composition further comprises a liquid emollient chosen from dimethicone, isopropyl isostearate, or mixtures thereof.

4. The skin care composition of claim 2, wherein the composition further comprises petrolatum and/or shea butter.

5. The skin care composition of claim 1, wherein the composition comprises from about 0.1 to about 5 wt. % of the fatty alcohol.

6. The skin care composition of claim 1, comprising at least two feel modifying particles comprising colloidal oatmeal and starch.

7. The skin care composition of claim 6, wherein the composition comprises from about 1.5 wt. % to about 4 wt. % of the corn starch and from about 0.1 to about 2 wt. % of the colloidal oatmeal.

8. The skin care composition of claim 1, wherein the composition comprises from about 6.5 to about 10 wt. % glycerin.

9. The skin care composition of claim 1, wherein the composition comprises 1.5% or less of the emulsifier.

10. The skin care composition of claim 9, wherein the composition comprises 1% or less of the emulsifier.

11. The skin care composition of claim 1, wherein the composition comprises an average tack force between 20 and 40 minutes of less than 750 g according to the Tack Method.

12. A skin care composition comprising:
a. a continuous phase comprising:
i. from about 55 to about 85 wt. % water;
ii. from about 5 to about 12 wt. % glycerin;
b. a dispersed phase comprising:
i. from about 1 to about 15 wt. % of at least two waxy emollients chosen from cetyl alcohol, glyceryl stearate, petrolatum, shea butter, or mixtures thereof;
ii. from about 3 to about 10 wt. % of a liquid emollient chosen from dimethicone, isopropyl isostearate, or mixtures thereof;
c. from about 0.1 to about 3 wt. % of colloidal oatmeal;
d. from about 0.1 to about 2 wt. % of an emulsifier comprising cetearyl olivate and sorbitan olivate.

13. The skin care composition of claim 12, wherein the composition comprises from about 6.5 to about 10 wt. % glycerin.

14. The skin care composition of claim 12, wherein the waxy emollients comprise cetyl alcohol, glyceryl stearate, and petrolatum.

15. The skin care composition of claim 14, wherein the waxy emollients further comprises shea butter and the composition comprises from about 8 to about 13 wt. % of the waxy emollient.

16. The skin care composition of claim 15, wherein the composition comprises from about 8% to about 10% glycerin.

17. The skin care composition of claim 16, further comprising niacinamide.

18. The skin care composition of claim 12, further comprising from about 1% to about 3% starch.

19. The skin care composition of claim 18, wherein the starch comprises modified corn starch.

20. The skin care composition of claim 12, comprising a % T of less than 50%.
